(19) United States
(12) Patent Application Publication
(10) Pub. No.: US 2020/0381108 A1
(43) Pub. Date: Dec. 3, 2020

(54) System and Method for Managing Diagnostic Imaging Costs

(71) Applicant: Premier Imaging Ventures, LLC, Columbus, OH (US)

(72) Inventors: Asif Ahmad, The Woodlands, TX (US); Ronald Alan Hosenfeld, II, Columbus, OH (US); Donald Renfrew, MD, Neenah, WI (US)

(21) Appl. No.: 16/983,537

(22) Filed: Aug. 3, 2020

Related U.S. Application Data
(63) Continuation-in-part of application No. 15/634,643, filed on Jun. 27, 2017, now abandoned.
(60) Provisional application No. 62/356,312, filed on Jun. 29, 2016.

(51) Int. Cl. G16H 40/20; G16H 30/20; G16H 15/00; G16H 70/20; G06Q 10/10; G06Q 40/00; G06Q 10/06; G06F 40/20

(52) U.S. Cl. CPC G16H 40/20; G16H 30/20; G16H 15/00; G06F 40/20; G06Q 10/10; G06Q 40/12; G06Q 10/06398; G16H 70/20

(57) ABSTRACT

An electronic medical record system monitors physician orders for diagnostic imaging and uses natural language processing to generate a field for characterizing the clinical significance of the image identified by the radiologist. This natural redundancy of evaluation of the efficacy of the imaging is used to generate reports allowing physicians to compare their effective use of diagnostic imaging against their peers.

### CROSS REFERENCE TO RELATED APPLICATION

This application is a continuation in part of U.S. application Ser. No. 15/634,643 filed Jun. 27, 2017, which claims the benefit of U.S. provisional application 62/356,312 filed Jun. 29, 2016, and hereby incorporated by reference.

### STATEMENT REGARDING FEDERALLY SPONSORED RESEARCH OR DEVELOPMENT

--

### BACKGROUND OF THE INVENTION

The present invention relates to electronic medical records and in particular to an electronic medical record system aiding in improved management of healthcare costs.

The increasing cost of healthcare presents a challenge in the delivery of high quality medical care. This challenge is being addressed, in part, by improved monitoring of the use of medical resources, such as drugs, medical tests, and the like, to ensure that these resources are used effectively.

Monitoring the use of medical resources in different clinical situations can be difficult. Resource use can be compared to mechanical guidelines, but these guidelines by their nature can easily oversimplify the subtleties of diagnosis and discourage proper healthcare delivery. The fast pace of medical innovation also makes it difficult to maintain detailed mechanical guidelines even for basic medical services.

Some of these problems can be avoided by using more general guidelines, but applying these general guidelines can require additional interpretation and judgment typically by a second physician, increasing costs and potentially putting an individual physician in an uncomfortable role of criticizing colleagues. Often, for this reason, general guidelines are interpreted by a committee, greatly increasing the cost of what is intended to be a cost-saving process.

In all cases, the oversight process itself tends to impose costs on every healthcare event, for example, in the form of additional physician time necessary to complete review documents, etc., that further offsets the cost reductions benefits intended to be provided. This is particularly true when wasteful practices, while costly, represent a small proportion of events.

### SUMMARY OF THE INVENTION

The present invention focuses on the delivery of radiological services and exploits a recognition by the inventors that unlike many medical resources, the delivery of diagnostic imaging normally involves cooperative effort by two healthcare professionals, for example, a primary care physician and a radiologist. This cooperative arrangement potentially provides an effective peer-level review to benchmark the effectiveness of a given diagnostic imaging procedure without the need for additional review by healthcare professionals or committees. In addition, a record system minimizes additional burdens and costs by the use of a natural language processing system that can work off the normal reporting done by a radiologist.

More specifically, the present invention employs natural language processing to attach a compact code to standard reports already generated by the radiologist. These compact codes capture the effectiveness of the imaging procedure with respect to the intended purpose of the procedure. Using this information, a detailed picture can be developed of the effectiveness of the delivery of diagnostic imaging services informed by professional insight without the overhead requiring study of the services by a third party or the mechanical application of a guideline. This information may be used in a variety of ways to improve utilization of diagnostic imaging services.

In one embodiment, the invention provides an electronic medical record system employing a digital network interconnecting set of nodes allowing the communication of medical information on the digital network between nodes for use by healthcare professionals in clinical decision-making. An electronic medical record server communicates with the set of nodes and operates to: (a) manage a database of the medical information accessible at the nodes; (b) record orders for diagnostic images, the orders indicating a clinical question motivating the order; (c) record position analysis of the diagnostic images obtained in response to the orders; (d) provide a natural language processing of at least one of the linked order and analysis to generate at least one of: a first result code indicating that the diagnostic images help resolve a clinical question and a second result code indicating that the diagnostic images do not help resolve the clinical question; and (e) generate a report indicating orders for diagnostic images characterized according to at least one of the first and second result codes.

It is thus a feature of at least one embodiment of the invention to make use of the natural redundancy of workflow in diagnostic imaging to provide a measure of effectiveness in that imaging such as can help contain rising medical costs. It is another feature of at least one embodiment of the invention to provide a system that can operate nearly invisibly to the standard radiology workflow through the use of natural language processing.

The report may indicate orders identified to at least one of the first and second result codes on a per physician basis.

It is thus a feature of at least one embodiment of the invention to help highlight physicians who are statistically less effective in the use of diagnostic imaging.

The physicians may be anonymous in the report.

It is thus a feature of at least one embodiment of the invention to provide a feedback mechanism to physicians that is not stigmatizing and which accommodates the fact that individual physician practices may differ.

The physicians maybe ranked according to numbers of orders characterized according to at least one of the first and second result codes.

It is thus a feature of at least one embodiment of the invention to provide a report that better illustrates not only the range of effectiveness among physicians but the extent to which an individual physician may be alone with respect to inefficient medical imaging resource usage.

The report may indicate either or both of absolute number of orders identified to at least one of the first and second result codes and a number of orders identified to at least one of the first and second result codes for a given physician in proportion to a total number of orders generated by the given physician.

It is thus a feature of at least one embodiment of the invention to provide both a measure of absolute resource usage and a measure that is calibrated to the number of images ordered by the physician.

The report may further identify at least one of a diagnostic image modality and a type of diagnostic image associated with each order.

It is thus a feature of at least one embodiment of the invention to detect trends that are associated with particular modalities (for example, CT) or particular types of images (for example, head CT) that may help reveal and explain usage variations.

The diagnostic orders may be linked to a third result code indicating a need for a follow-up diagnostic image and the electronic medical record server may further generate a second report indicating compliance in obtaining the follow-up diagnostic image.

It is thus a feature of at least one embodiment of the invention to provide an automatic method of ensuring proper follow-up imaging through a simple coding process by the radiologist.

The electronic medical record server may further generate a second report indicating a fiscal expenditure for diagnostic images according to the orders identified to at least one of the first and second result codes.

It is thus a feature of at least one embodiment of the invention to provide a report clearly indicating costs of procedures to help provide cost-effective use of diagnostic imaging.

The diagnostic orders may be further linked to an additional result code indicating diagnostic imaging associated with an interventional procedure and diagnostic imaging associated with medical emergencies.

It is thus a feature of at least one embodiment of the invention to provide a method of filtering out types of diagnostic imaging that are less susceptible to efficiency measures.

These particular objects and advantages may apply to only some embodiments falling within the claims and thus do not define the scope of the invention.

### BRIEF DESCRIPTION OF THE DRAWINGS

FIG. 1 is a perspective view of a simplified electronic medical record system for managing workflow and image transmission with respect to a diagnostic imaging suite;

FIG. 2 is a simplified diagram of an electronic medical record including a coding provided by the present invention;

FIG. 3 is a software flowchart and data flowchart showing the data collection and analysis points of the present invention in applying natural language processing to the development of i-code scoring of radiographic procedures;

FIG. 4 is an example generalized report prepared from the data collected by the present system;

FIG. 5 is an example specific report showing CT head scans per physician identified to the clinical significance of the scans by that physician;

FIG. 6 is an example specific report showing CT head scan usage broken out per physician with respect to the total number of scans;

FIG. 7 is a figure similar to that of FIG. 5 showing CT head scans associated with a diagnosis of headache again identified to the clinical significance of the scans by that physician;

FIG. 8 is a figure similar to that of FIG. 5 showing chest x-ray image orders identified as to clinical significance with respect to a diagnosis of cough, shortness of breath, and chest pain;

FIG. 9 is a figure similar to FIG. 6 showing chest x-rays per physician with respect to the total number of x-rays;

FIG. 10 is an example specific report showing recommended additional imaging studies for follow-up before and after physician feedback using the report system of the present invention; and

FIG. 11 is a detailed flow chart of the process of generating i-codes using the natural language processing of FIG. 3.

### DETAILED DESCRIPTION OF THE PREFERRED EMBODIMENT

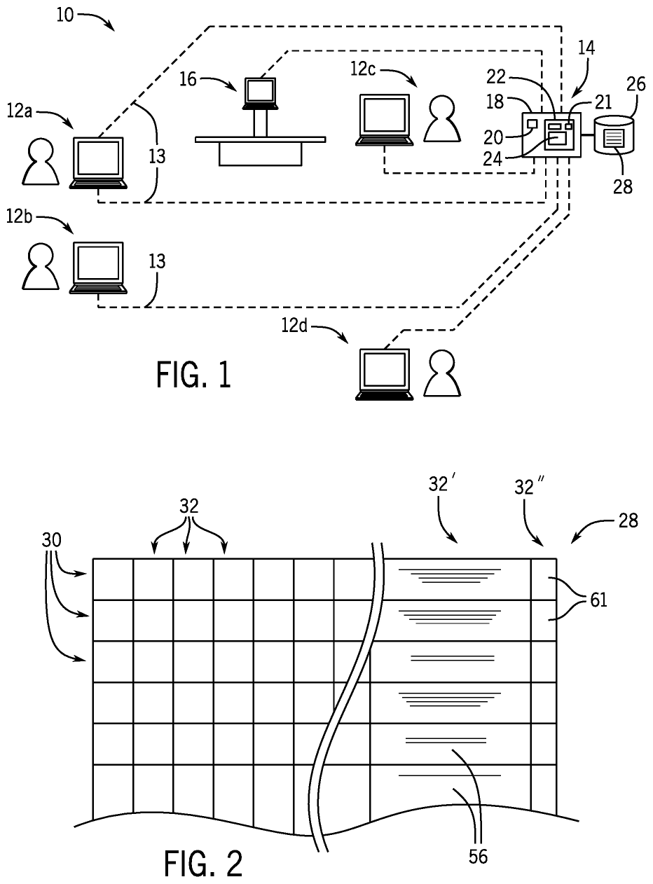

Referring now to FIG. 1, an electronic medical record system 10 may provide electrical communication with and between multiple communication nodes 12a-12d. The communication nodes 12 include data terminals (desktop computers or mobile devices) for use by healthcare professionals for access to an electronic medical record (EMR) server 14 holding comprehensive data on patients in a healthcare setting. An electronic medical record as used herein should be understood to include all records necessary for the normal practice of a healthcare facility including those which provide any or all of patient demographic data, clinical data related to those patients including diagnostic images and image archiving, as well as billing and discharge information. Generally, each of these nodes 12 will provide for an electronic processor executing a stored program to receive user input, for example, through a keyboard or the like and provide user output to a display screen. The nodes 12 will further include network interface circuitry for communicating over a network 13.

The EMR server 14 may also communicate directly or indirectly with medical instruments including diagnostic imaging equipment 16 such as ultrasound, x-ray, CT, MRI, PET, or nuclear medicine machines. The diagnostic imaging equipment 16 may receive orders and transfer image data, for example, in DICOM form, over the network 13 as communicated between the diagnostic imaging equipment 16 and the various communication nodes 12 and the EMR server 14. As is generally understood in the art, the network 13 may provide for digital communication over wired and/or wireless links, for example, the latter employing high-speed Ethernet.

The EMR server 14, like each of the communication nodes 12, may provide for an electronic computer 18 having one or more processors 20 communicating with a memory 22 holding a stored program 24 whose operation will be described below. Part of the stored program 24 may manage a database 26 holding an electronic medical record EMR file 28 providing for medical record data and images for a large number of patients. In addition, part of the stored program 24 may implement a natural language processing system 21, for example, using the Natural Language Tool Kit (NLTK). This toolkit provides a Python platform for the construction of natural language processing as described in Bird, Steven, Edward Loper and Ewan Klein (2009), Natural Language Processing with Python. O'Reilly Media Inc. hereby incorporated by reference.

Referring now to FIG. 2, the EMR file 28 may be logically represented, for example, as a flat file having multiple rows 30 each identified to a particular patient and multiple columns providing fields 32 keyed to the patient. Example fields 32 of the EMR file 28 include a patient identification number, patient demographic information, and billing information for the patient as well as reports and diagnostic information about the patient's medical care including diagnoses, orders for lab tests and diagnostic imaging, pharmacy orders and the like, the latter dated and identified to particular physicians treating the patient.

With respect to diagnostic imaging orders, one field 32′ may permit a text or recorded speech entry, for example, dictated by a radiologist, memorializing analysis of a diagnostic image associated with a diagnostic imaging order, the latter of which may also be incorporated into the EMR file 28. As will be discussed in greater detail, the invention adds an additional text field 32″ allowing the natural language processing system 21 to operate on the field 32′ to provide a code summarizing field 32′ for later machine analysis and follow through.

Referring again to FIG. 1, generally the communication nodes 12 will include one or more primary physician nodes 12a and 12b being most simply network-connected desktop computers allowing physicians to record data about the patient, for example, from an office visit, in the EMR file 28 and review information about the patient from the EMR file 28 as well as to communicate with other healthcare professionals on the system, for example, to request diagnostic imaging via a diagnostic imaging order.

The terminals provided by the communication nodes 12 may also include a radiologist node 12c for use by a radiologist for access to the EMR file 28 to receive diagnostic imaging orders and to record images obtained from the diagnostic imaging equipment 16 obtained in response to those orders. The radiologist node 12c allows the radiologist to enter analysis of any obtained diagnostic images (recorded in field 32′) and a coding (recorded in field 32″) related to those images, linked to the patient, as we discussed below. As part of this process, the diagnostic imaging equipment 16 communicates with the EMR file 28 to augment information associated with the obtained images and to provide images directly to the EMR file 28 or to cooperate in the storage of those images.

One terminal may provide an administrator node 12d for use by hospital administrators to review information collected by the present system to better assess the use of diagnostic imaging.

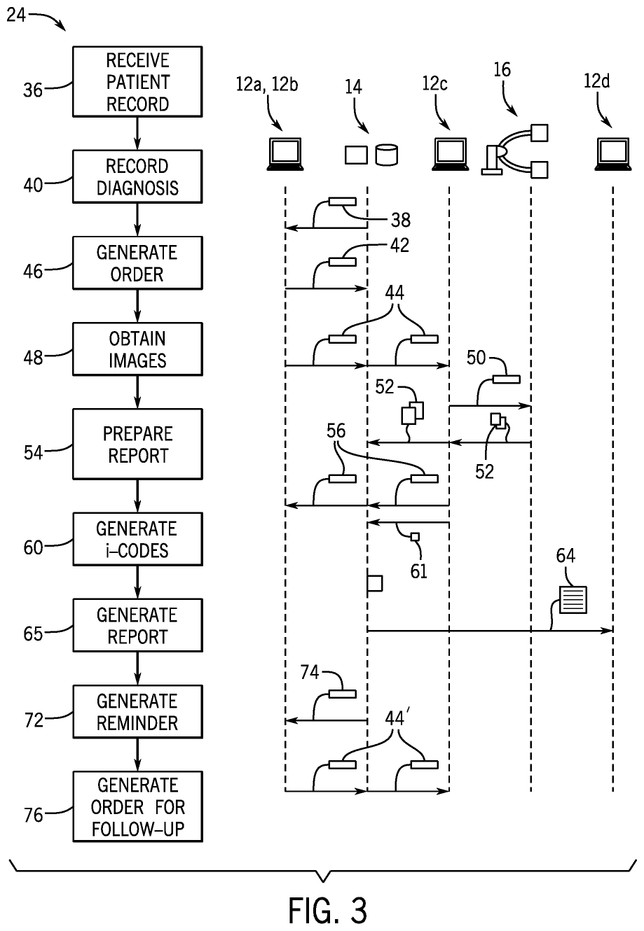

Referring now to FIG. 3, the stored program 24 of the EMR server 14, operating in conjunction with programming in each of the communication nodes 12, enables a workflow starting, as indicated by process block 36, by a receipt of patient records at a physician node 12a or 12b, for example, initiated in response to an office visit by a patient or other diagnostic event. In this process, medical record data 38 may be forwarded from the EMR server 14 to the physician node 12a or 12b for review of that information in the manner of a paper record.

Based on a review of that medical record data 38 and other information available to the physician, the physician may prepare a diagnosis and report 42 as indicated at process block 40. This report 42 may include observations and new diagnostic conclusions that are returned to the server for enrollment in the EMR file 28. As part of generating this report 42, the physician may also generate an order 44, as indicated by process block 46, requesting a diagnostic imaging procedure, for example, a CT scan. The term order as used herein should be understood to refer to any communicated data related to the processing of a specific diagnostic imaging procedure including, for example, billing codes and the like. This order 44 will be associated with a clinical condition of interest to guide the radiologist in the selection of procedures, contrast agents, and the like. The order may equally provide for diagnostic imaging other than CT including, for example, other x-ray procedures, ultrasound, MRI, PET, nuclear medicine scans and the like such as may be interpreted by a radiologist. In one embodiment, the order 44 may include a diagnosis code (the condition for which the scan is to be obtained) and image type (e.g., chest x-ray), a date of the order, and a modality of the diagnostic imaging equipment 16 (e.g., x-ray, CT, MRI).

The order 44 is then forwarded to the EMR server 14 for recording in the EMR file 28 which operates to forward the order 44 to a radiologist at radiologist node 12c.

As indicated by process block 48, in response to the order 44, the radiologist may obtain the necessary images using the diagnostic imaging equipment 16. In this regard, the radiologist may input control and patient information 50 to the diagnostic imaging equipment 16 and/or the EMR server 14, for example, identifying x-ray technique, the number of films, orientation of the machine, etc.

Upon completion of the imaging procedure, the radiologist may receive image data 52 which is also forwarded to the EMR file 28 at the EMR server 14.

The complex process of interpreting diagnostic images is normally undertaken by the radiologist as indicated by process block 54. In this process, the radiologist reviews the orders of the physician generating the original order 44 to interpret the image data 52 in light of the intended diagnostic purpose of the diagnostic images. In addition, the radiologist may provide a general review of the images for other possible medical conditions. All of this information is recorded by the radiologist as a report 56 stored in the field 32′ of the EMR file 28, usually in the form of a text or speech file, providing a human understandable text narrative.

The report 56 is also provided to the natural language processor 21 of the EMR server 14 which, as indicated by process block 60, generates an i-code 61 stored in field 32″ of the EMR file 28 summarizing particular points about the review in a machine-readable form. This i-code may link to ICD-9 or ICD 10 codes or CPT codes and comprise a prefix letter followed by one or more alphanumeric characters as follows:

TABLE I

| i-code | Option | Explanation |
|---|---|---|
| i.0 | CT, US, XR, MR, NM | indicates a need for additional |
| | | imaging to be performed |
| | | immediately of the modality |
| | | indicated in the option. |
| i.1 | NA | Indicates a negative or |
| | | noncontributory result of the |
| | | imaging examination, that is, |
| | | no imaging finding on the |
| | | examination to account for the |
| | | submitted clinical question or |
| | | history provided by the |
| | | ordering physician. |
| i.2 | | indicates a positive result or |
| | | finding on the imaging |
| | | examination, that is, a clear |
| | | finding on the imaging study |
| | | to account for the submitted |
| | | clinical question or history |
| | | provided by the ordering |
| | | physician. |
| i.3 | CT, US, XR, MR, NM | indicates need for additional |
| | Follow-up date | imaging at a later date of the |
| | (weeks/months) | modality indicated in the |
| | | option. An additional option |
| | | indicates a date of the follow- |
| | | up imaging in weeks or |
| | | months. |
| i.4 | | indicates that the imaging |
| | | study was associated with an |
| | | interventional procedure. |
| i.5 | | indicates the study is a |
| | | “critical result” typically |
| | | associated with medical |
| | | emergencies. |

These i-codes may be used individually or in combination. An i.0 code, for example, might be used if the current imaging study is ambiguous or points to a problem better diagnosed in a different modality.

An i.1 code generally indicates that the diagnostic imaging did not confirm the source of the clinical problem. For example, a response to a CT scan for abdominal pain might indicate “no evidence or cause of abdominal pain is present” in the resulting image.

An i.2 code for the same CT scan for abdominal pain, in contrast, would indicate “there is a cause of abdominal pain evident” in the diagnostic images.

The i.3 code, which indicates the need for non-urgent additional imaging, might take the form of i.3.MR.6, for example, which would indicate that an MRI scan is necessary for this patient in six months or i.3.XR.12 which would indicate that an x-ray is a necessary for this patient in one year.

The i.4 code is used for interventional procedures such as a biopsy, angiogram, or injection. These are typically not diagnostic studies, but rather reflect the use of diagnostic imaging guidance to perform the interventional procedure and accordingly cannot be characterized as to whether they confirm or fail to confirm a particular diagnosis.

The i.5 code may be used in critical situations such as intracranial bleeding, pulmonary embolus, acute stroke, ruptured intra-abdominal viscous, etc. where evaluation of efficient use of medical imaging is equally important to ensure sufficient care level for life threatening situations.

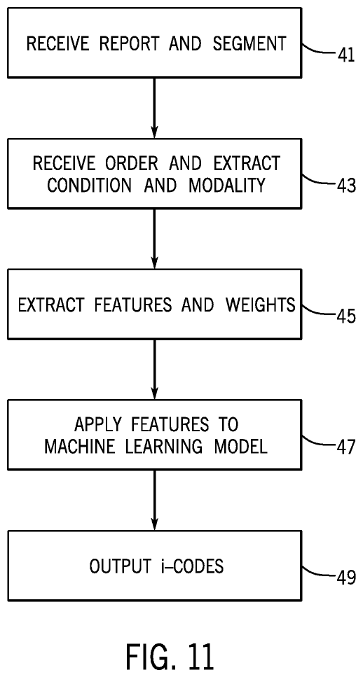

Referring now to FIG. 11, in one embodiment, the natural language processor 21 may receive the report 56 that has been prepared using a standard template provided to the physicians for this purpose, the template having specific delineated sections such as: History, Comparisons, Findings, Impression, Conclusions, and Recommendations, intended to encourage the physician to include certain information in the report 56 as is standard practice. In this regard, the template may incorporate one or more standardized text “signatures” in each section to aid in the segmentation of the report 56 by machine. For example, the signatures may provide for standardized titles, meta-text, phrases or the like. At process block 41, the report 56 is received and segmented into the various sections using the standardized signatures to extract a text block including all of the text of the Impression/Conclusion/Recommendation sections. Such machine separation identifies the signatures and extracts text between the appropriate signatures.

At succeeding process block 43, the order 44 linked to the report 56 may also be received and “scraped” using standard text processing procedures to identify and extract a clinical condition of interest justifying the radiology procedure associated with the report 56 and the intended modality and forming the foundation for imaging described in the report 56. This extraction may, for example, search by pattern matching the billing codes, ICD-9 codes, or the like or may look for particular sections of the order 44 and identify keywords that indicate the clinical condition being analyzed, the modality being used for the imaging, and possibly other objective qualities of the imaging such as contrast media, etc. This information may alternatively or in addition be extracted from the reports 56.

At process block 45, based on the clinical condition being analyzed, a set of pre-prepared relevant terms are identified within the report 56 related to the clinical condition and the modality of the radiographic procedure. These relevant terms may be automatically identified in the report 56, for example, by comparing a list of such relevant terms from a lookup table indexed according to the clinical condition and modality using pattern matching to the extracted information from the report 56.

Generally, the relevant terms are selected to include phrases used by physicians indicative of the qualities measured by the i-codes. Thus, for example, if the clinical conditions indicated at process block 43 relate to respiratory problems, the relevant terms may include “pneumonia,” “pulmonary nodule,” and “pneumothorax.” Or, for example, with a clinical condition related to upper right quadrant pain, the relevant terms may include “cholecystitis” or “intraperitoneal air.” Generally, a comprehensive but different set of relevant terms will be provided for each clinical condition and modality covering both expected non-critical diagnoses for the clinical condition as well as critical diagnoses. Terms associated with critical diagnoses may include, for example, “acute fracture” as related to spinal conditions, or “hematoma” as related to head injury, or “acute appendicitis” or “hemorrhage” related to lower abdomen, or “free air” related to abdomen work, or “ectopic” as related to ultrasound examinations.

This list of relevant terms linked to clinical conditions and modalities by a lookup table may be prepared manually with input from experienced physicians and may be selected from a concordance of words extracted from a test set of reports 56 described below to remove common stop words. Each of these relevant terms maybe subject to lemmatization and stemming to ensure broad scope of this list of relevant terms. Note, at this time, the relationship of these relevant terms to the ultimate i-codes need not be precisely identified.

In an iterative process, the relevant terms may be tested against a sample set of documents using frequency-inverse document frequency for each relevant term, the frequency-inverse document frequency being a weight that increases proportionally to the number of times a word appears in a document normalized by the rarity of that word. Relevant terms with lower weighting may be discarded in favor of relevant terms with higher weighting according to a threshold. The established weights may be used in a supervised machine learning extraction of i-codes (as will be described in more detail below), for example, by providing only those relevant terms with the highest weights to the machine learning process or providing other algorithmically dependent weighting approaches in the machine learning algorithm.

In a proof of concept of the invention, the inventors used a corpus of 7090 radiology reports 56 taken from actual medical practice, eliminating common stop words and lemmatizing the remaining terms. Each report 56 of this corpus was then manually tagged with one or more i-codes (and possibly the options described above in table I), applied to the feature extraction of process blocks 41, 43, and 45 described above, and used 80% for training a machine learning algorithm of the natural language processor 21 and 20% for testing that training. The result was a trained natural language processor 21.

At process block 47, the extracted features from the report 56 and 44 are applied to the trained machine learning model of the natural language processor 21 to produce i-code values per process block 49 without manual intervention.

The following provide some more specific examples of the application of these i-codes.

### Example 1

Consider a patient who has obtained a CT scan for abdominal pain through the emergency room, but the scan shows no acute intra-abdominal findings. However, the patient was noted to have an indeterminant nodule in the lungs, and requires a follow-up CT scan in nine months. This study would be coded: i.1, i.3.CT.9.

### Example 2

Consider the setting of a patient who has obtained a chest x-ray for cough. The chest x-ray demonstrates pneumonia, but also shows a small pulmonary nodule that requires an immediate CT scan for work up. This study would be coded: i.2, i.0.CT.

### Example 3

Consider the setting of a patient who obtains an ultrasound for right upper quadrant pain. Examination shows acute cholecystitis, but also evidence for free intraperitoneal air suggesting perforation. This is a medical emergency. The study would be coded: i.2, i.5.

The i-codes 61 together with the coded reports 56 are then returned to the EMR server 14 and enrolled in the EMR file 28 for the appropriate patient. The coded reports 56 are also forwarded to the physician originally posting the diagnostic imaging order 44.

Referring still to FIG. 3, as indicated by process block 65, the EMR file 28 may be reviewed at the EMR server 14 to generate a utilization report 64 based on the i-codes 61. At this time, or on a separate schedule, the EMR server 14 may also generate follow-up information as will be discussed below. The generation of the utilization report 64 indicated by process block 65 includes the step of forwarding the utilization report 64 to the administrator node 12d for evaluation of the utilization of diagnostic imaging resources and consideration of methods of improving this utilization. Because the system of the present invention enlists the skilled knowledge of radiologists who are already involved with and knowledgeable about matter that they are coding, the utilization report provides high quality assessments at relatively low additional cost.

As will be discussed below, the system may measure follow-up when an i.3 code is entered and compliance with follow-up recommendations can also be measured.

As indicated by process block 72, the EMR server 14 may further use the i-codes 61 to develop a follow-up schedule for future diagnostic imaging procedures. In one embodiment, the EMR server 14 executing the program 24 may send reminders 74 to the physicians at appropriate future times when a follow-up has been indicated. At process block 76, subsequent diagnostic imaging orders 44′ (similar to those generated at process block 46) responsive to those follow-up reminders may be captured by the EMR server 14 in order to generate statistics on compliance with follow-up recommendations. Identifying the subsequent orders that match requests for follow-up that are indicated by i-codes 61 can be done automatically based on the identities of the patient and orders for the same diagnostic imaging procedure within a predetermined time or may be indicated by the physician as being responsive to the follow-up. An option may also exist for the physician to indicate that the follow-up has been considered and provide reasons that a follow-up is not considered medically necessary.

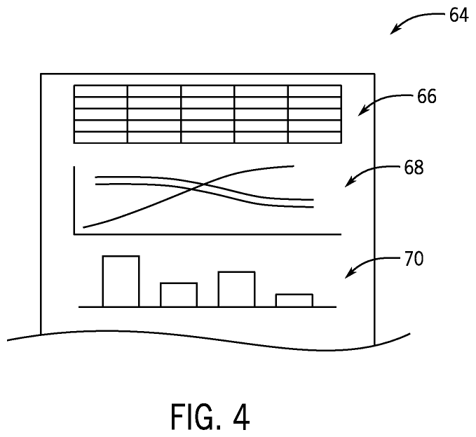

Referring now also to FIG. 4, the utilization report 64 may provide a variety of different types of information. For example, the utilization report 64 may provide a ranked listing of physicians 66 who have generated diagnostic imaging orders 44 over a fixed period of time and can indicate numerically and/or by ranking the number of reports 56 resulting from those orders 44 that are coded i.1, that is, instances where the imaging examination does not find evidence supporting the clinical question for which the examination was intended. When quantified, this information may be an absolute number, a number relative to a proportion of diagnostic imaging procedures ordered by the physician or department or, for example, a ratio of studies that are coded i.1 (don't confirm the diagnosis) to studies that are coded i.2 (confirm the diagnosis). Each physician's compliance with follow-up orders may also be indicated in the same or a different listing, distinguishing, for example, between situations of compliance with follow-up recommendations, noncompliance, and recordation of a subsequent conclusion that a follow-up is not required. Finally, a report may be generated indicating the number of additional scans requested by the radiologists under code i.0.

In addition, or alternatively, a trend line 68 over a predetermined time interval may be developed for various measures, for example, number of procedures coded i.1 sorted by department or physician, procedures coded i.2, total procedures, follow-up compliance and the like.

In one version, the utilization report 64 may provide a fiscal measure 70 indicating, for example, total expenditures for diagnostic imaging procedures, for example, sorted into various categories with respect to the i-codes 61. For example, a bar chart may be provided having bars for cost of medical imaging procedures having i.1 coding and a separate category for i.2 coding together with total diagnostic imaging costs. More generally the portion of these two categories can be compared to each of the other categories indicating the relative significance of the categories with i.1 coding.

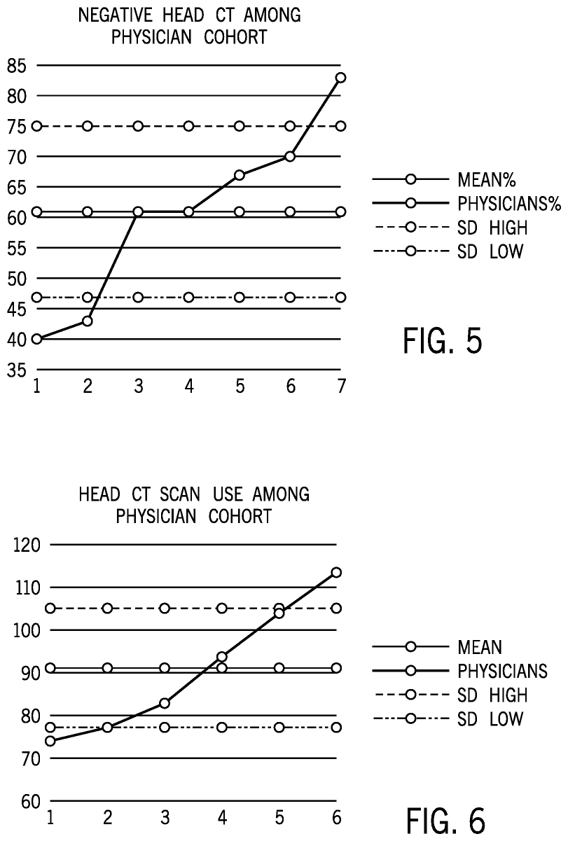

Referring now to FIG. 5, one example report may show diagnostic imaging using the modality of CT and the image type of head CT for a period of time for seven anonymous emergency room physicians at a single hospital. This report shows the percentage of CT head scans having been coded i.1 and thus failing to answer the clinical question with respect to possible head injury. This report represents actual medical data and shows a wide range in the percentage of negative scans among these physicians. In one case, nearly 85 percent of all scans are negative. This would imply that one or more of these physicians needs to do a better job of screening their patients before subjecting them to CT had scans. A mean and upper and lower standard deviation value is marked by the horizontal lines in each of these example reports.

Referring now to FIG. 6, a similar report may simply list the number of CT head scans ordered during a given period of time without regard to the clinical significance of those scans. Again, there is a substantial range in the number of CT scans ordered which may provide useful feedback to the physician.

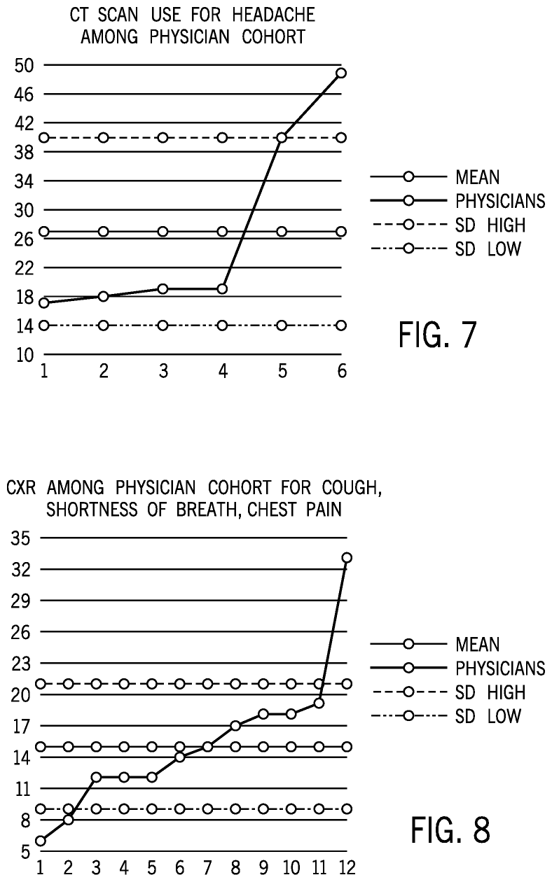

Referring now to FIG. 7 a similar report for CT head scans may be filtered to identify those for the particular diagnosis of headache providing more precision with respect to the meaning of a negative outcome.

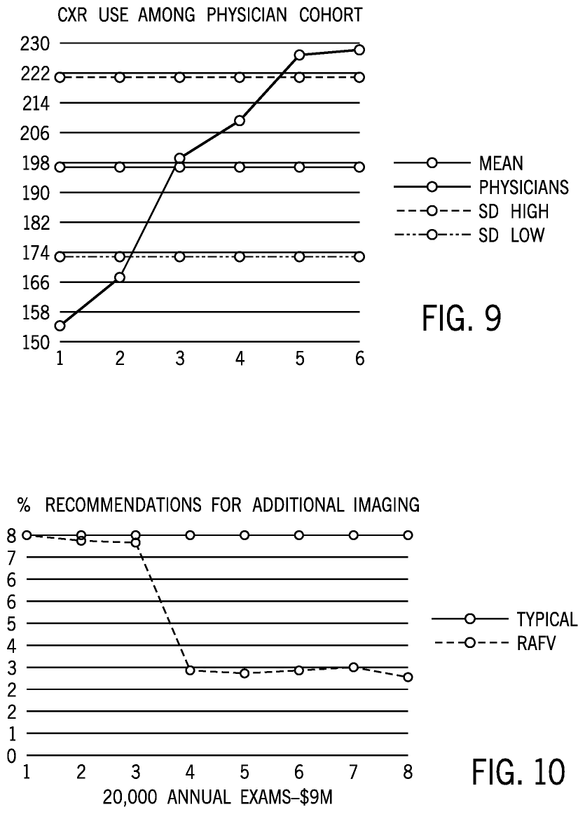

FIG. 8 shows a similar report generated with respect to chest x-rays used by emergency room physicians with the diagnosis of cough, shortness of breath, or chest pain showing total number of x-rays ordered during a historical period. FIG. 9 shows a comparable report not limited to a particular diagnosis of FIG. 8. Again, these charts show wide variation in imaging utilization that can help guide physicians with respect to their own practice.

FIG. 10 shows a monthly average number of diagnostic images coded as i.3 as a function of month where those diagnostic images indicate a need for additional imaging studies as a follow-up. Feedback to the physicians occurring at month 3 significantly reduced the number of imaging studies by 20,000 annual exams, saving 9 million dollars for the healthcare system.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

References to “a computer” and “a processor” or “the microprocessor” and “the processor,” can be understood to include one or more microprocessors that can communicate in a stand-alone and/or a distributed environment(s), and can thus be configured to communicate via wired or wireless communications with other processors, where such one or more processor can be configured to operate on one or more processor-controlled devices that can be similar or different devices. Furthermore, references to memory, unless otherwise specified, can include one or more processor-readable and accessible memory elements and/or components that can be internal to the processor-controlled device, external to the processor-controlled device, and can be accessed via a wired or wireless network.

It will be appreciated that the nodes 12 represent logical nodes and that they are not necessary located at a particular location but may follow the designated individuals through a variety of devices.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties.

What we claim is:

1. An electronic medical record system comprising:
a digital network;
a set of nodes allowing a communication of medical information on the digital network between nodes for use by healthcare professionals in clinical decision-making; and
an electronic medical record server communicating with the set of nodes and operating to:
(a) manage a database of the medical information accessible at the nodes;
(b) record orders for diagnostic images, the orders indicating a clinical question motivating the order,
(c) record position analysis of the diagnostic images obtained in response to the orders;
(d) provide a natural language processing of at least one of the linked order and analysis to generate at least one of: a first result code indicating that the diagnostic images help resolve clinical question and a second result code indicating that the diagnostic images do not help resolve the clinical question; and
(d) generate a report indicating orders for diagnostic images characterized according to at least one of the first and second result codes.

2. The electronic medical record system of claim 1 wherein the report indicates orders identified to at least one of the first and second result codes on a per physician basis.

3. The electronic medical record system of claim 2 wherein physicians generating orders for diagnostic imaging are anonymous in the report.

4. The electronic medical record system of claim 2 wherein physicians generating orders for diagnostic imaging are ranked according to numbers of orders characterized according to at least one of the first and second result codes.

5. The electronic medical record system of claim 1 wherein the report is selected from the group consisting of: a report indicating absolute number of orders identified to at least one of the first and second result codes and a report indicating a number of orders identified to at least one of the first and second result codes for a given physician in proportion to a total number of orders generated by the given physician.

6. The electronic medical record system of claim 1 wherein the report further identifies at least one of a diagnostic image modality and a type of diagnostic image associated with each order.

7. The electronic medical record system of claim 1 wherein the diagnostic orders are further linked to a third result code indicating a need for a follow-up diagnostic image and wherein the electronic medical record server further generates a second report indicating compliance in obtaining the follow-up diagnostic image.

8. The electronic medical record system of claim 1 wherein the electronic medical record server further generates a second report indicating a fiscal expenditure for diagnostic images according to the orders identified to at least one of the first and second result codes.

9. The electronic medical record system of claim 1 wherein the diagnostic orders are further linked to at least one additional result code selected from the group of codes indicating: diagnostic imaging associated with an interventional procedure and diagnostic imaging associated with medical emergencies.

10. A method of providing efficient utilization of medical imaging using an electronic medical record system having a digital network interconnecting a set of nodes exchanging medical information for use by healthcare professionals in clinical decision-making, the nodes communicating with an electronic medical record server executing a stored program to perform the steps of:
(a) manage a database of the medical information accessible at the nodes;
(b) record orders for diagnostic images, the orders indicating a clinical question motivating the order,
(c) link the orders to both an analysis of diagnostic images obtained in response to the orders and at least one of: a first result code indicating that the diagnostic images help resolve clinical question and a second result code indicating that the diagnostic images do not help resolve the clinical question; and
(d) generate a report indicating orders for diagnostic images characterized according to at least one of the first and second result codes.

11. The method of claim 10 wherein the report indicates orders identified to at least one of the first and second result codes on a per physician basis.

12. The method of claim 11 wherein physicians generating orders for diagnostic imaging are anonymous in the report.

13. The method of claim 12 wherein physicians generating orders for diagnostic imaging are ranked according to numbers of orders characterized according to at least one of the first and second result codes.

14. The method of claim 10 wherein the report is selected from the group consisting of: a report indicating absolute number of orders identified to at least one of the first and second result codes and a report indicating a number of orders identified to at least one of the first and second result codes for a given physician in proportion to a total number of orders generated by the given physician.

15. The method of claim 10 wherein the report further identifies at least one of a diagnostic image modality and a type of diagnostic image associated with each order.

16. The method of claim 10 wherein the diagnostic orders are further linked to a third result code indicating a need for a follow-up diagnostic image and wherein the electronic medical record server further generates a second report indicating compliance with obtaining the follow-up diagnostic image.

17. The method of claim 10 wherein the electronic medical record server further generates a second report indicating a fiscal expenditure for diagnostic images according to the orders identified to at least one of the first and second result codes.

18. The method of claim 10 wherein the diagnostic orders are further linked to at least one additional result code selected from the group of codes indicating: diagnostic imaging associated with an interventional procedure and diagnostic imaging associated with medical emergencies.
